(19) United States
(12) Patent Application Publication
(10) Pub. No.: US 2023/0338134 A1
(43) Pub. Date: Oct. 26, 2023

(54) Suture-free stent grafts and stent graft constructs with integrated extensions and methods for forming

(71) Applicant: The Secant Group, LLC, Telford, PA (US)

(72) Inventors: Amanda K. Weber, Macungie, PA (US); Peter D. Gabriele, Frisco, TX (US); Andrew Metzger, Lafayette Hill, PA (US)

(21) Appl. No.: 18/445,111

(22) Filed: Apr. 11, 2023

Related U.S. Application Data
(60) Provisional application No. 63/362,766, filed on Apr. 11, 2022.
(60) Provisional application No. 63/362,762, filed on Apr. 11, 2022.

(51) Int. Cl. A61F 2/07; D03D 3/02; D03D 1/00; D03D 15/283; D03D 11/00

(52) U.S. Cl. CPC A61F 2/07; D03D 3/02; D03D 1/00; D03D 15/283; D03D 11/00; A61F 2002/075; D10B 2509/00; D10B 2331/04; D10B 2509/06

(57) ABSTRACT

A suture-free stent graft is disclosed including a woven graft tube having woven extensions extending from an outer face of the woven graft tube. Each of the woven extensions is integrally woven with the woven graft tube such that warp from the woven graft tube is de-interlaced from at a first end and re-interlaced at a second end. Supplemental warp is interlaced into the woven graft tube in replacement of the deinterlaced warp so as to maintain weave density in the woven graft tube under the woven extensions. The woven extensions and portions of the woven graft tube disposed directly under the woven extension have independent weft from one another. A suture-free stent graft construct is disclosed including the suture-free stent graft and at least one stent wire disposed about the suture-free stent graft, at least partially disposed between the woven graft tube and the woven extensions at securement sites.

### RELATED APPLICATIONS

This application claims the benefit of and priority to U.S. Provisional Patent Application No. 63/362,766, filed Apr. 11, 2022, entitled “Suture-Free Stent Grafts and Stent Graft Constructs with Integrated Extensions and Methods for Forming,” and U.S. Provisional Patent Application No. 63/362,762, filed Apr. 11, 2022, entitled “Articles, Compositions, and Methods for Sutureless Implants,” which are hereby incorporated by reference in their entirety.

### FIELD OF THE INVENTION

This application is directed to suture-free stent grafts and stent graft constructs and methods for forming suture-free stent grafts and stent graft constructs. More particularly, this application is directed to suture-free stent grafts and stent graft constructs and methods for forming suture-free stent grafts and stent graft constructs having integrated extensions.

### BACKGROUND OF THE INVENTION

Stent grafts are important abluminal cardiovascular device constructs important to both structural heart assignments such as transcatheter heart valves as well as aortic and vascular stenting. However, the state of the art is primitive and depends on suture ligation to permanently fix the textile graft to the stenting scaffold. Suture ligation for fixing a textile graft to a stenciling scaffold has many disadvantages, including: a high economic burden for the manual suturing process, abluminal vascular wall trauma and aortic wall injury (including scratches, abrasions, endoleakage, aortic wall dissention, aneurysms, infections, abluminal intimal hyperplasia, fibrosis, wall stiffening, inflammation, disruption of hemodynamic flow, and nickel/metal leaching), abrasion and scuffing of the stent-graft sutures with the endothelial abluminal surface during deployment and in service.

There is a need for stent grafts which do not suffer from these disadvantages.

### BRIEF DESCRIPTION OF THE INVENTION

In one exemplary embodiment, a suture-free stent graft includes a woven graft tube having an inner face and an outer face and a plurality of woven extensions extending from the outer face of the woven graft tube. Each of the plurality of woven extensions is integrally woven with the woven graft tube at a first end and a second end such that warp from the woven graft tube is de-interlaced from the woven graft tube at the first end and re-interlaced with the woven graft tube at the second end. Supplemental warp is interlaced into the woven graft tube in replacement of the deinterlaced warp so as to maintain weave density in the woven graft tube under the plurality of woven extensions. The plurality of woven extensions and portions of the woven graft tube disposed directly under the plurality of woven extension have independent weft from one another.

In another exemplary embodiment, a suture-free stent graft construct includes a suture-free stent graft and at least one stent wire disposed about the suture-free stent graft. The suture-free stent graft includes a woven graft tube having an inner face and an outer face and a plurality of woven extensions extending from the outer face of the woven graft tube. Each of the plurality of woven extensions is integrally woven with the woven graft tube at a first end and a second end such that warp from the woven graft tube is de-interlaced from the woven graft tube at the first end and re-interlaced with the woven graft tube at the second end. Supplemental warp is interlaced into the woven graft tube in replacement of the deinterlaced warp so as to maintain weave density in the woven graft tube under the plurality of woven extensions. The plurality of woven extensions and portions of the woven graft tube disposed directly under the plurality of woven extension have independent weft from one another. The at least one stent wire is at least partially disposed between the woven graft tube and the plurality of woven extensions at a plurality of securement sites, securing the woven graft tube to the at least one stent wire.

Further aspects of the subject matter of the present disclosure are provided by the following clauses:

A suture-free stent graft includes a woven graft tube having an inner face and an outer face and a plurality of woven extensions extending from the outer face of the woven graft tube. Each of the plurality of woven extensions is integrally woven with the woven graft tube at a first end and a second end such that warp from the woven graft tube is de-interlaced from the woven graft tube at the first end and re-interlaced with the woven graft tube at the second end. Supplemental warp is interlaced into the woven graft tube in replacement of the deinterlaced warp so as to maintain weave density in the woven graft tube under the plurality of woven extensions. The plurality of woven extensions and portions of the woven graft tube disposed directly under the plurality of woven extension have independent weft from one another.

The suture-free stent graft of any preceding clause, wherein the suture-free stent graft is free of floating yarns forming the plurality of woven extensions.

The suture-free stent graft of any preceding clause, wherein each of the plurality of woven extensions has only woven edges except where interlaced and integral with the woven graft tube.

The suture-free stent graft of any preceding clause, wherein the plurality of woven extensions include loop extensions integral with the woven graft tube at only the first end and the second end, defining a plurality of through-channels.

The suture-free stent graft of any preceding clause, wherein the plurality of woven extensions include slot extensions integral with the woven graft tube at the first end and the second end as well as along one edge extending from the first end to the second end, defining a plurality of pockets.

The suture-free stent graft of any preceding clause, wherein the woven graft tube is formed of 20-40 denier yarn and includes at least 150 ends per inch and at least 100 picks per inch throughout the woven graft tube.

The suture-free stent graft of any preceding clause, wherein the 20-40 denier yarn is polyethylene terephthalate yarn.

The suture-free stent graft of any preceding clause, wherein the plurality of woven extensions is positioned along and about, and oriented relative to, the woven graft tube so as to receive at least one stent wire having a predetermined shape.

A suture-free stent graft construct includes a suture-free stent graft and at least one stent wire disposed about the suture-free stent graft. The suture-free stent graft includes a woven graft tube having an inner face and an outer face and a plurality of woven extensions extending from the outer face of the woven graft tube. Each of the plurality of woven extensions is integrally woven with the woven graft tube at a first end and a second end such that warp from the woven graft tube is de-interlaced from the woven graft tube at the first end and re-interlaced with the woven graft tube at the second end. Supplemental warp is interlaced into the woven graft tube in replacement of the deinterlaced warp so as to maintain weave density in the woven graft tube under the plurality of woven extensions. The plurality of woven extensions and portions of the woven graft tube disposed directly under the plurality of woven extension have independent weft from one another. The at least one stent wire is at least partially disposed between the woven graft tube and the plurality of woven extensions at a plurality of securement sites, securing the woven graft tube to the at least one stent wire.

The suture-free stent graft construct of any preceding clause, wherein the plurality of woven extensions include loop extensions integral with the woven graft tube at only the first end and the second end, defining a plurality of through-channels, and the at least one stent wire passes through the plurality of through-channels.

The suture-free stent graft construct of any preceding clause, wherein the plurality of woven extensions include slot extensions integral with the woven graft tube at the first end and the second end as well as along one edge extending from the first end to the second end, defining a plurality of pockets, and the at least one stent wire is partially disposed within the plurality of pockets.

### BRIEF DESCRIPTION OF THE DRAWINGS

FIG. 1 is a planar simulation of a woven graft tube with a woven extension weave pattern, according to an embodiment of the present disclosure.

FIG. 2 is a profile simulation of the woven graft tube of FIG. 1, according to an embodiment of the present disclosure.

FIG. 3 is a woven loomstate diagram of the woven graft tube of FIG. 1, according to an embodiment of the present disclosure.

FIG. 4 is a planar simulation of a woven graft tube with a woven extension weave pattern having a 1:1 weft ratio for the woven graft tube to the woven extension, according to an embodiment of the present disclosure.

FIG. 5 is a profile simulation of the woven graft tube of FIG. 4, according to an embodiment of the present disclosure.

FIG. 6 is a woven loomstate diagram of the woven graft tube of FIG. 4, according to an embodiment of the present disclosure.

FIG. 7 is a profile simulation of a woven graft tube with a woven extension weave pattern having a 2:1 weft ratio for the woven graft tube to the woven extension, according to an embodiment of the present disclosure.

FIG. 8 is a woven loomstate diagram of the woven graft tube of FIG. 7, according to an embodiment of the present disclosure.

FIG. 9 is an illustration of a suture-free stent graft including loop extensions as the woven extensions, forming through-channels, according to an embodiment of the present disclosure.

FIG. 10 is an illustration of a suture-free stent graft including slot extension as the woven extensions, forming pockets (the dashed lines indicating the placement of stent wires within the pockets), according to an embodiment of the present disclosure.

FIG. 11 is an illustration of a suture-free stent graft having a plurality of loop extensions integral with the woven graft tube, according to an embodiment of the present disclosure.

FIG. 12 is an illustration of a suture-free stent graft configured to accept a plurality of stents distributed axially along the woven graft tube secured by loop extension as the woven extensions, forming through-channels through which stent wires passes through, according to an embodiment of the present disclosure.

FIG. 13 is an illustration of a stent in the form of a single wire continuous wire scaffold configured to propagate axially along the woven graft tube, according to an embodiment of the present disclosure.

FIG. 14 is an illustration of a single wire continuous wire scaffold stent propagating axially along a woven graft tube and passing through loop extensions as the woven extensions, according to an embodiment of the present disclosure.

FIG. 15 is an illustration of a suture-free stent graft with woven extensions in the cross-machine direction of the loom, according to an embodiment of the present disclosure.

FIG. 16 is a schematic illustrating bonding of a stent wire formed of nitinol to a PET substrate, according to an embodiment of the present disclosure.

FIG. 17 is a suture-free stent graft as woven, according to an embodiment of the present disclosure.

FIG. 18 is the suture-free stent graft of FIG. 17 after finishing, according to an embodiment of the present disclosure.

FIG. 19 is a suture-free stent graft construct formed by threading a stent wire through the securement sites of the suture-free stent graft of FIG. 18, according to an embodiment of the present disclosure.

FIG. 20 is a suture-free stent graft with circumferential pockets, according to an embodiment of the present disclosure.

Wherever possible, the same reference numbers will be used throughout the drawings to represent the same parts.

### DETAILED DESCRIPTION OF THE INVENTION

Unless otherwise specified, “about” indicates a variance of ±5% from the value being modified.

Embodiments of the present invention provide suture-free stent grafts and stent graft constructs and methods for forming suture-free stent grafts and stent graft constructs with integrated extensions. Embodiments of the present invention, in comparison to existing solutions lacking at least one of the features disclosed herein, represent a more advanced approach than known solutions for stent grafts and stent graft constructs and demonstrate a break away from traditional engineering designs and the advancement of interfacial surface bonding technologies to reduce or eliminate the dangers of knotted ligature in stent grafts, reduce or eliminate nickel leaching, reduce or eliminate fibrotic immune system response, or combinations thereof Further, embodiments of the present invention, in comparison to existing solutions lacking at least one of the features disclosed herein, may extend the service stent grafts, including, but not limited to, aortic stents, by reducing a patient's natural response to foreign materials and trauma.

Textile interfaces currently using ligatures may be replaced with textile grafts of polyethylene terephthalate (“PET”), polyvinylidene fluoride (“PVDF”), ultra-high molecular weight polyethylene (“UHMWPE”), polyethersulfone (“PES”), polypropylene (“PP”), polytetrafluoroethylene (“PTFE”), thermoplastic polyurethane (“TPU”), or combinations thereof, alone or in combination with polymeric biodegradable glycerol ester chemistries (poly(glycerol sebacate) (“PGS”), poly(glycerol-co-sebacate) acrylate (“PGSA”), poly(glycerol sebacate urethane) (“PGSU”), poly(glycerol sebacate)-co-poly(glycolic acid) (“PGS-co-PGA”), poly(glycerol sebacate)-co-poly(lactic acid) (“PGS-co-PLA”)) as bonding agents for textile interfaces where ligatures exist.

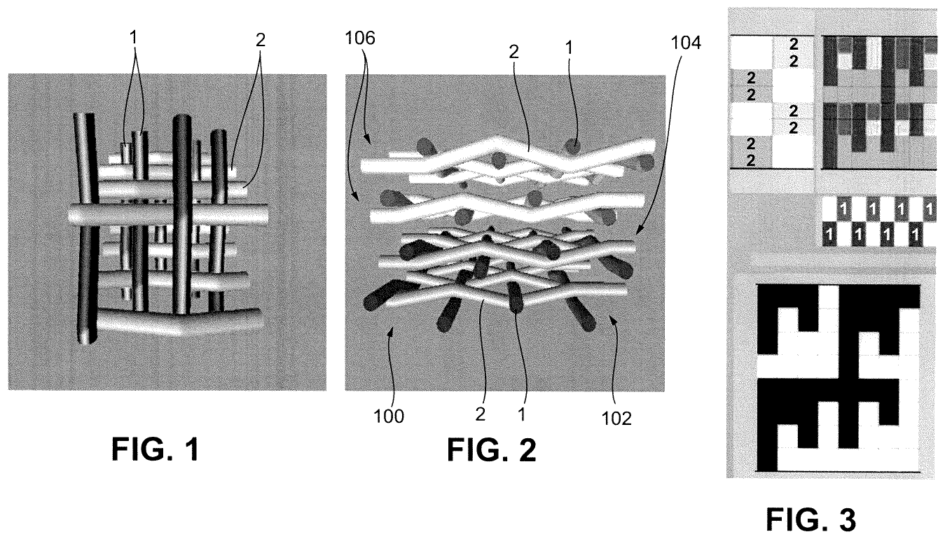

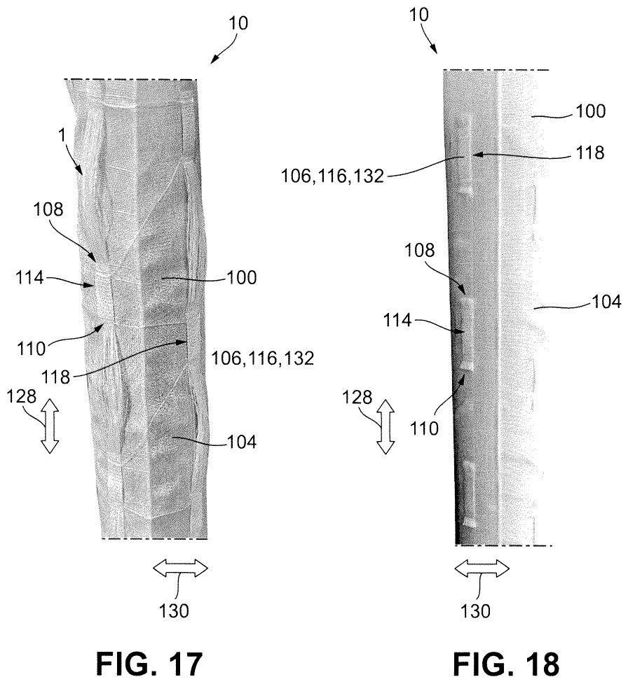

Referring to FIGS. 1-15, in one embodiment, a suture-free stent graft 10 includes a woven graft tube 100 having an inner face 102 and an outer face 104 and a plurality of woven extensions 106 extending from the outer face 104 of the woven graft tube 100. Each of the plurality of woven extensions 106 is integrally woven with the woven graft tube 100 at a first end 108 and a second end 110 such that warp 1 from the woven graft tube 100 is de-interlaced from the woven graft tube 100 at the first end 108 and re-interlaced with woven graft tube 100 at the second end 110. Supplemental warp 1 is interlaced into the woven graft tube 100 in replacement of the deinterlaced warp 1 so as to maintain weave density in the woven graft tube 100 under the plurality of woven extensions 106 (as seen in the experimental embodiment in FIG. 17). The plurality of woven extensions 106 and portions of the woven graft tube 100 disposed directly under the plurality of woven extensions 106 have independent weft 2 from one another.

Adding the supplemental warp 1 may maintain a targeted liquid tightness in the woven graft tube 100 by counteracting the decreased density of warp 1 that are being lifted off the woven graft tube 100 to form the plurality of woven extensions 106. In a comparative example which is otherwise identical but without adding the supplemental warp 1, the density of the woven graft tube 100 where the warp 1 has been lifted away to form the plurality of woven extensions 106 would be decreased, increasing porosity and leakage, and the resulting floating warp 1 or weft 2, particularly warp 1 or weft 2 that would not be interlaced, may lead to snagging or pulling of the ends or picks as a stent wire 112 is threaded through the suture-free stent graft 10.

FIGS. 1-3 illustrate planar (FIG. 1) and profile (FIG. 2) simulations of a woven graft tube 100 and a woven extension 106, and a loomstate (FIG. 3) for the weave patterns thereof When woven on a shuttle loom, the weft 2 of the woven graft tube 100 weaves a top and bottom seamless woven graft tube 100 independent of the weft 2 of the plurality of woven extensions 106, which forms a separate interwoven layer. These simulations show an open pocket for the woven extension 106 weave, but this may be altered so the woven extension 106 weave is bound, interlaced, or tacked to the woven graft tube 100 at various locations.

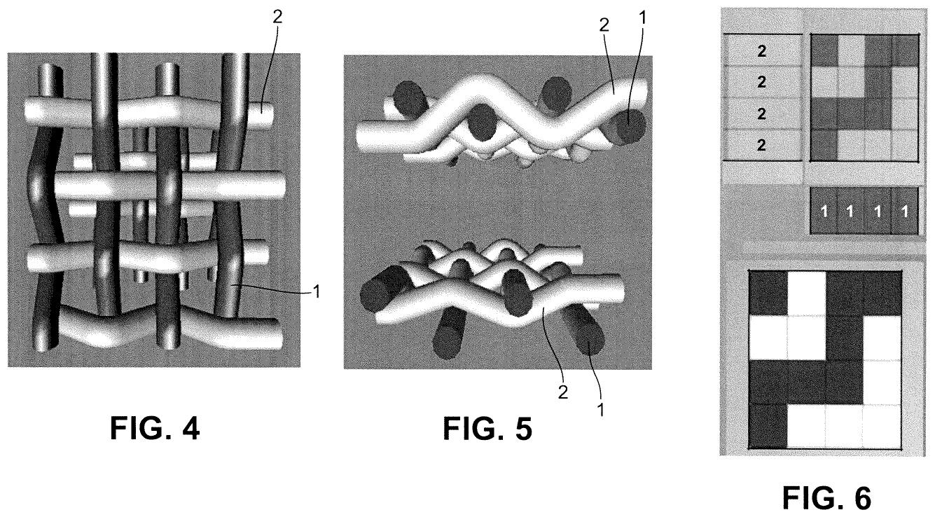

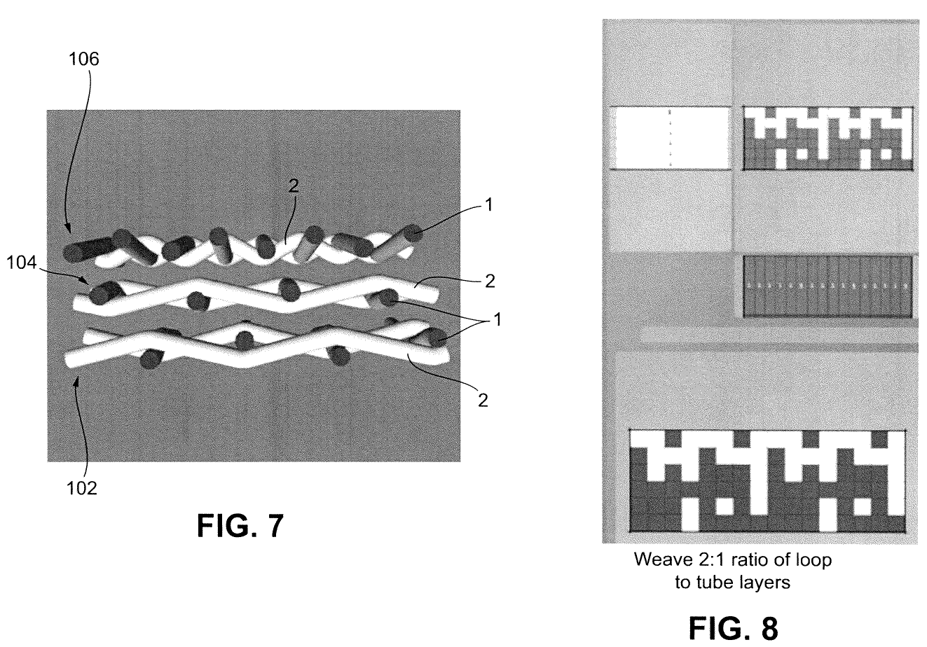

FIGS. 4-8 illustrate planar (FIG. 4) and profile (FIGS. 5 and 7) simulations of a woven graft tube 100, and loomstates for the (FIGS. 6 and 8) for the weave patterns thereof These woven simulations indicate one of the many custom ways in which the woven graft tube 100 may be formed. In further embodiments, additional layers may be formed with extensions 106 being formed on both the outer face 104 and the inner face 102 of the woven graft tube 100. The simulations show woven extensions on one face of the woven graft tube 100. The suture-free stent graft 10 may be woven with a 1:1 weft 2 ratio for the woven graft tube 100 to the woven extension 106 (FIGS. 4-6), but may have up to a 4:1, alternatively 3:1, alternatively 2:1 (FIGS. 7-8) weft ratio for the woven extension 106 to the woven graft tube 100 for different woven effects in the layers.

The suture-free stent graft 10 may be free of floating yarns forming the plurality of woven extensions 106. Each of the plurality of woven extensions 106 may have only woven edges 114 except where interlaced and integral with the woven graft tube 100.

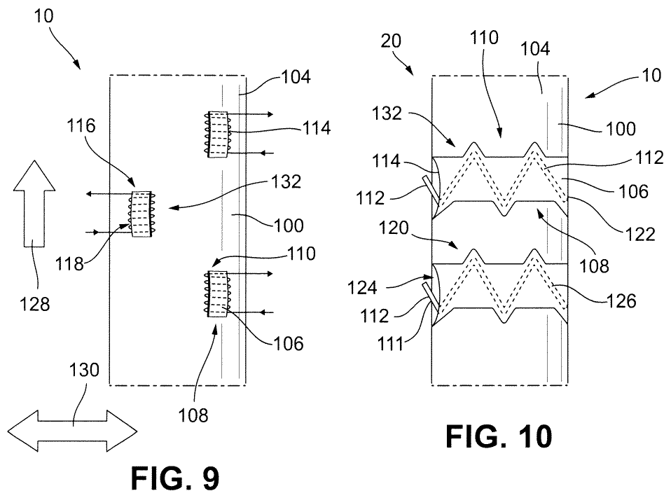

Referring to FIG. 9, in one embodiment, the plurality of woven extensions 106 includes loop extensions 116 integral with the woven graft tube 100 at only the first end 108 and the second end 11, defining a plurality of through-channels 118.

Referring to FIG. 10, in another embodiment the plurality of woven extensions 106 includes slot extensions 120 integral with the woven graft tube 100 at the first end 108 and the second end 110 as well as along one edge 122 extending from the first end 108 to the second end 110, defining a plurality of pockets 124.

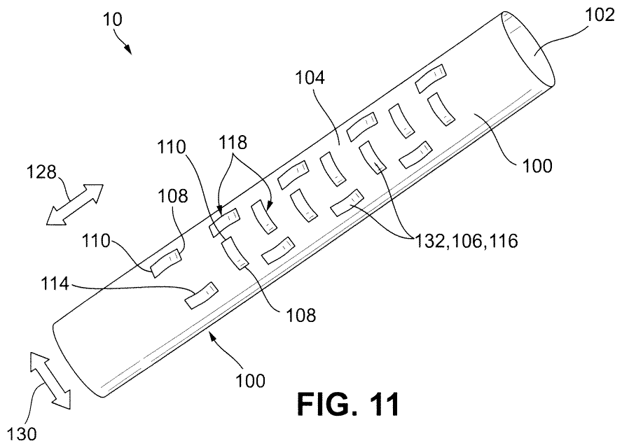

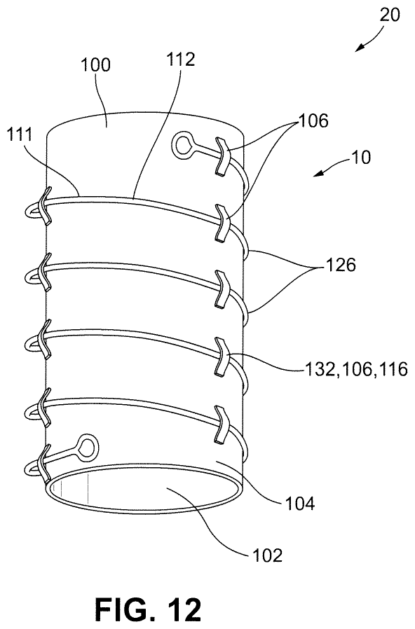

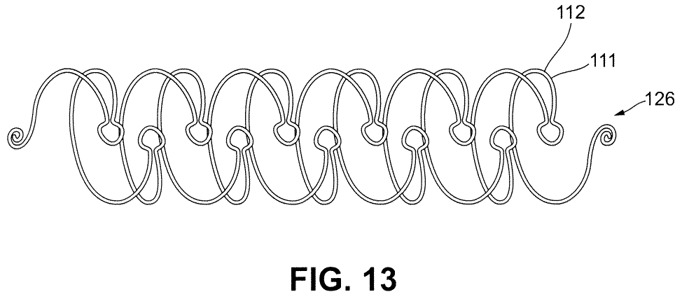

Referring to FIGS. 11, 12, and 20, the plurality of woven extensions 106 may be positioned along and/or about, and oriented relative to, the woven graft tube 100 so as to receive a least one structural reinforcement 111, such as, but not limited to, at least one stent wire 112 (FIG. 12), at least one annular support structure 113 (FIG. 20), having a predetermined shape. The annular support structure 113 may be any suitable structure, including, but not limited to, a plastic ring, a metal ring, a molded ring, a curable polymer ring, stent ring, or combinations thereof.

Referring to FIG. 12, one suture-free stent graft 10 may include a plurality of stents 126 distributed axially along the woven graft tube 100. The suture-free stent graft 10 may be configured to accept the plurality of stents 126 distributed axially along the woven graft tube 100 secured by loop extensions 116 as the woven extensions 106, forming through-channels 118 through which the stents 126 pass through.

Referring to FIGS. 13-15, alternatively, the one suture-free stent graft 10 may include a single stent 126 in the form of a single stent wire 112 that propagates axially along the woven graft tube 100. In one embodiment (FIG. 13), the stent 126 in the form of a single stent wire 112 continuous wire scaffold configured to propagate axially along the woven graft tube 100. The single stent wire 112 continuous wire scaffold stent may propagate axially along the woven graft tube 100 while passing through loop extensions 116 as the woven extensions 106.

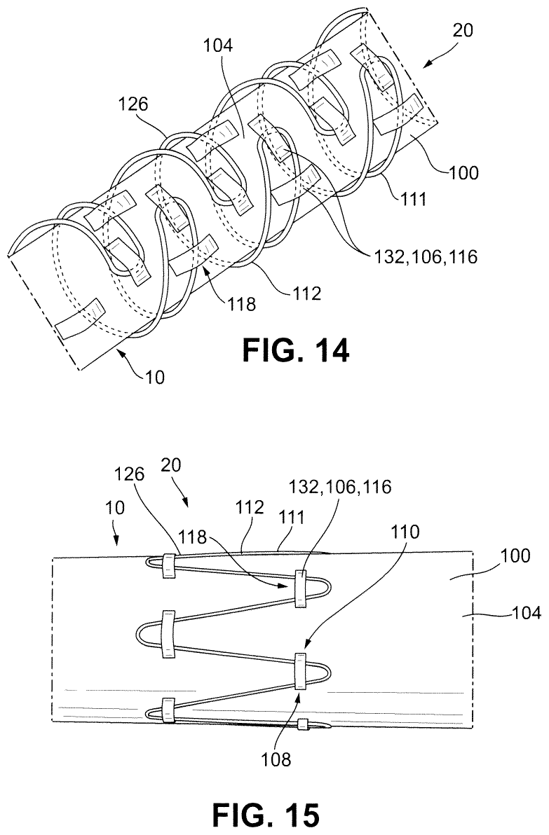

The woven extension 106 may be oriented in the cross-machine direction 130 of the loom (FIG. 15) or in the machine direction 128 (FIG. 9), or both (FIGS. 11 and 14).

The woven graft tube 100 may be formed of any suitable yarn, including, but not limited to, 16-44 denier yarn, alternatively 16-24 denier yarn, alternatively 20-28 denier yarn, alternatively 24-32 denier yarn, alternatively 28-36 denier yarn, alternatively 32-40 denier yarn, alternatively 36-44 denier yarn, alternatively 20-40 denier yarn, alternatively 16 denier yarn, alternatively 20 denier yarn, alternatively 40 denier yarn, or any subrange or combination thereof The yarn may have any suitable composition, including, but not limited to, PET, PVDF, UHMWPE, PES, PP, PTFE, TPU, or combinations thereof.

The plurality of woven extensions 106 may be formed of any suitable yarn, including, but not limited to, 16-44 denier yarn, alternatively 16-24 denier yarn, alternatively 20-28 denier yarn, alternatively 24-32 denier yarn, alternatively 28-36 denier yarn, alternatively 32-40 denier yarn, alternatively 36-44 denier yarn, alternatively 20-40 denier yarn, alternatively 16 denier yarn, alternatively 20 denier yarn, alternatively 40 denier yarn, or any subrange or combination thereof. The yarn may have any suitable composition, including, but not limited to, PET.

In one embodiment, the woven graft tube 100 includes at least 100 ends per inch and at least 100 picks per inch throughout the woven graft tube, alternatively 150 ends per inch and at least 100 picks per inch, alternatively at least 200 ends per inch and at least 150 picks per inch, alternatively 100-350 ends per inch and 100-240 picks per inch, alternatively 100-200 ends per inch and 100-150 picks per inch, alternatively 150-250 ends per inch and 125-175 picks per inch, alternatively 200-300 ends per inch and 150-200 picks per inch, alternatively 250-350 ends per inch and 175-240 picks per inch, or any sub-range or combination thereof.

In one embodiment, the plurality of woven extensions 106 includes at least 50 ends per inch and at least 50 picks per inch throughout the plurality of woven extensions, alternatively at least 150 ends per inch and at least 100 picks per inch, alternatively at least 200 ends per inch and at least 150 picks per inch, alternatively 50-350 ends per inch and 50-240 picks per inch, alternatively 50-150 ends per inch and 50-110 picks per inch, alternatively 100-200 ends per inch and 80-140 picks per inch, alternatively 150-250 ends per inch and 110-170 picks per inch, alternatively 200-300 ends per inch and 140-200 picks per inch, alternatively 250-350 ends per inch and 170-240 picks per inch or any sub-range or combination thereof.

Referring to FIGS. 12, 14, and 15, in one embodiment, a suture-free stent graft construct 20 includes the suture-free stent graft 10 and at least one stent wire 112 disposed about the suture-free stent graft 10. The at least one stent wire 112 is at least partially disposed between the woven graft tube 100 and the plurality of woven extensions 106 at a plurality of securement sites 132, securing the woven graft tube 100 to the at least one stent wire 112. When the plurality of woven extensions 106 includes loop extensions 116 integral with the woven graft tube 100 at only the first end 108 and the second end 110, defining a plurality of through-channels 118, the at least one stent wire 112 may pass through the plurality of through-channels 118. When the plurality of woven extensions 106 includes slot extensions 120 integral with the woven graft tube 100 at the first end 108 and the second end 110 as well as along one edge 122 extending from the first end 108 to the second end 110, defining a plurality of pockets 124, the at least one stent wire 112 is partially disposed within the plurality of pockets 124.

The stent wire 112 may be made from any suitable material, including, but not limited to, a nickel-titanium alloy (“nitinol”) having a nickel:titanium alloy of about 4:5 to about 2:3.

The woven extensions 106 may be secured in place over the at least one stent wire 112 by any suitable adherent system, including, but not limited to, polymeric biodegradable glycerol esters (PGS, PGSA, PGSU, PGS-co-PGA, PGS-co-PLA, or combinations thereof), chelating polymers (such as, but not limited to, catechol-modified polymeric biodegradable glycerol ester), primers, bonding agents, or combinations thereof. The adherent system may also passivate the at least one stent wire 112. The adherent system may be characterized by elastomeric interfacial bonding between the at least one stent wire 112 and the underlying woven graft tube 100.

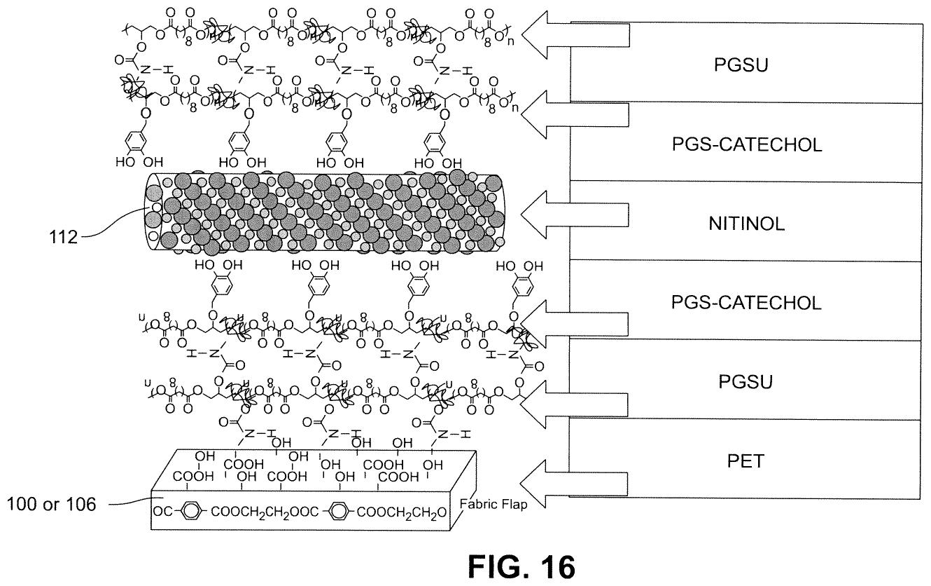

Referring to FIG. 16, in one embodiment, the at least one stent wire 112 is chemically bonded to at least one of the woven graft tubes 100 and the plurality of woven extensions 106 using polymeric biodegradable glycerol ester passivators and surface modification of at least one of the woven graft tube 100 or the plurality of woven extensions 106. In a further embodiment, the at least one stent wire 112 is chemically bonded to both the woven graft tube 100 and the plurality of woven extensions 106. By way of non-limiting example, a nitinol stent wire 112 may be coated with PGS-catechol, which may be bonded to a layer of PGSU, which in turn may be bonded to PET of the woven graft tube 100, the plurality of woven extensions 106, or both.

In one embodiment, a method for forming a suture-free stent graft 10 includes weaving a multilayered, multifilament graft 10 including a woven graft tube 100 and a plurality of woven extensions 106 extending from an outer face 104 of the woven graft tube 100. Each of the plurality of woven extensions 106 is integrally woven with the woven graft tube 100 at a first end 108 and a second end 110 such that warp 1 from the woven graft tube 100 is de-interlaced from the woven graft tube 100 at the first end 108 and re-interlaced with the woven graft tube 100 at the second end 110. Supplemental warp 1 is interlaced into the woven graft tube 100 in replacement of the deinterlaced warp 1 so as to maintain weave density in the woven graft tube 100 under the plurality of woven extensions 106. The woven graft tube 100 and the plurality of woven extensions 106 have independent weft 2 from one another. The plurality of woven extensions 106 protrudes over the woven graft tube 100 and create a through-channel 118 or pocket 124 of fabric independent of the outer face 104 of the woven graft tube 100 such that the through-channel 118 or pocket 124 shares no common warp 1 or weft threads 2 with the woven graft tube 100 directly below the through-channel 118 or pocket 124. In a further embodiment, the suture-free stent graft 10 is formed using a shuttle loom that may form clean uncut woven edges 114 on the woven graft tube 100 and the plurality of woven extensions 106 which mitigates fraying in the machine direction 128. In order to increase the protrusion of the plurality of woven extensions 106 above the woven graft tube 100 (and thereby enhance the separation), the ratio of weft 2 to warp 1 may be increased, the size of the weft 2 yarn may be increased, a different material, such as, but not limited to, monofilament yarn may be added or substituted, or combinations thereof.

### EXAMPLES

Referring to FIGS. 17-19, a suture-free stent graft 10 was formed with a weaving sequence on a shuttle loom, and then combined with a stent wire to form a suture-free stent graft construct 20.

In a first step, shuttle B was engaged to weave the top tubular ground for a minimum of two picks then shuttle C was engaged to weave only with supplemental warp on the front left side of the tube for a minimum of one pick. This sequence was repeated until the desired length of loop was formed (four picks of shuttle B to two picks of shuttle C in the present example). In a second step, shuttle B was engaged to weave the tubular warp ends and the supplemental warp that form the back face, left loop to form an attachment of loop to the back tubular ground for a minimum of four picks. Shuttle B continued to weave to interlace the top tubular warp ends and supplemental warp of the front face, left loop for a minimum of four picks. In a third step, shuttle B was engaged to weave the tubular ground for a minimum of two picks and then shuttle C was engaged to weave only with the supplemental warp on the back left face of tube for a minimum of one pick and repeat this step until desired length of loop was woven (four picks of shuttle B to two picks of shuttle C in the present example). In a fourth step, shuttle B was engaged to weave the tubular warp ends and the supplemental warp that form the front face, right loop to form an attachment of loop to top tubular ground for a minimum of four picks. Shuttle B continued to weave to interlace the back tubular warp ends and supplemental warp of the back face, left loop for a minimum of four picks. In a fifth step, shuttle B was engaged to weave the tubular ground for a minimum of two picks then shuttle A was engaged to weave only with the supplemental warp on the front face right side of the tube for a minimum of one pick. This sequence was repeated until the desired length of loop was formed (four picks of shuttle B to two picks of shuttle A in the present example). In a sixth step, shuttle B was engaged to weave the tubular warp ends and the supplemental warp that form the back face right loop to form an attachment of loop to the back tubular ground for a minimum of four picks. Shuttle B continued to weave to interlace the top tubular warp ends and supplemental warp of the front face, right loop for a minimum of four picks. In a seventh step, shuttle B was engaged to weave the tubular ground for a minimum of two picks then shuttle A was engaged to weave only with the supplemental warp on the back face right side of the tube for a minimum of one pick. This sequence was repeated until the desired length of loop was formed (four picks of shuttle B to two picks of shuttle A in the present example). In an eighth step, shuttle B was engaged to weave the tubular warp ends and the supplemental warp that form the front face left loop to form an attachment of loop to the top tubular ground for a minimum of four picks. Shuttle B continued to weave to interlace the back tubular warp ends and supplemental warp of the back face, right loop for a minimum of four picks. Although in this example, the supplemental warp was floating on the top face of the suture-free stent graft 10, the supplemental warp may also float behind the top tubular face within the woven graft tube 100 itself. Similarly, the entire suture-free stent graft may be woven inside-out.

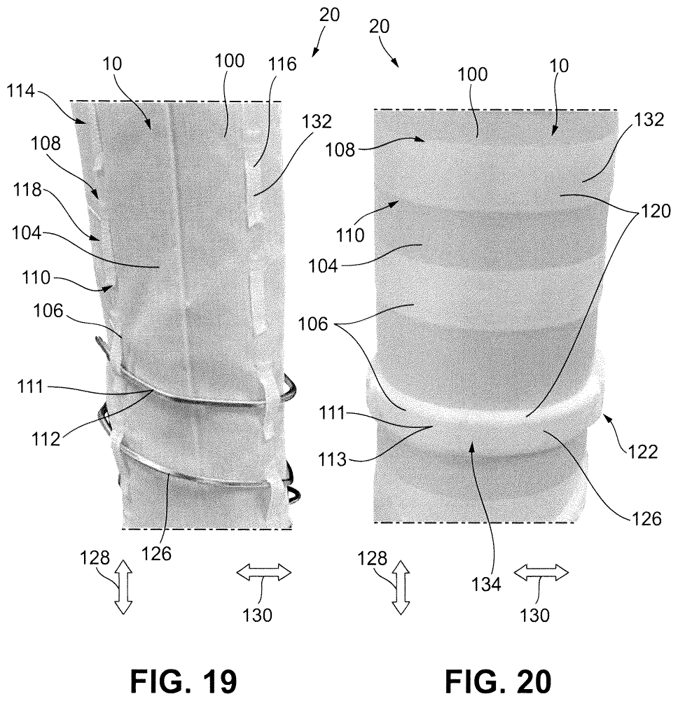

The suture-free stent graft 10 as woven in this example is shown in FIG. 17. FIG. 18 shows the suture-free stent graft of FIG. 17 after finishing. FIG. 19 shows the suture-free stent graft construct 20 formed by threading a stent wire 112 (helical) through the securement sites 132 of the suture-free stent graft 10. Finishing may include, but is not limited to, heat treating the suture-free stent graft 10 so as to fix final dimensions and shape of the suture-free stent graft 10, trimming supplemental warp ends floating over the outer face 104 of the woven graft tube 100, or combinations thereof.

FIG. 20 shows another exemplary embodiment in which a structural reinforcement 111 was inserted into a circumferentially-oriented slot extension 120, and then the edge 122 of which was joined to the opening of the pocket 124 so as to form a circumferential pocket 134.

While the foregoing specification illustrates and describes exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A suture-free stent graft, comprising:
a woven graft tube having an inner face and an outer face; and
a plurality of woven extensions extending from the outer face of the woven graft tube, wherein:
each of the plurality of woven extensions is integrally woven with the woven graft tube at a first end and a second end such that warp from the woven graft tube is de-interlaced from the woven graft tube at the first end and re-interlaced with the woven graft tube at the second end;
supplemental warp is interlaced into the woven graft tube in replacement of the deinterlaced warp so as to maintain weave density in the woven graft tube under the plurality of woven extensions, and
the plurality of woven extensions and portions of the woven graft tube disposed directly under the plurality of woven extension have independent weft from one another.

2. The suture-free stent graft of claim 1, wherein the suture-free stent graft is free of floating yarns forming the plurality of woven extensions.

3. The suture-free stent graft of claim 1, wherein each of the plurality of woven extensions has only woven edges except where interlaced and integral with the woven graft tube.

4. The suture-free stent graft of claim 1, wherein the plurality of woven extensions include loop extensions integral with the woven graft tube at only the first end and the second end, defining a plurality of through-channels.

5. The suture-free stent graft of claim 1, wherein the plurality of woven extensions include slot extensions integral with the woven graft tube at the first end and the second end as well as along one edge extending from the first end to the second end, defining a plurality of pockets.

6. The suture-free stent graft of claim 1, wherein the woven graft tube is formed of 20-40 denier yarn and includes at least 150 ends per inch and at least 100 picks per inch throughout the woven graft tube.

7. The suture-free stent graft of claim 6, wherein the 20-40 denier yarn is polyethylene terephthalate yarn.

8. The suture-free stent graft of claim 1, wherein the plurality of woven extensions are positioned along and about, and oriented relative to, the woven graft tube so as to receive at least one stent wire having a predetermined shape.

9. A suture-free stent graft construct, comprising:
a suture-free stent graft, comprising:
a woven graft tube having an inner face and an outer face; and
a plurality of woven extensions extending from the outer face of the woven graft tube,
wherein:
each of the plurality of woven extensions is integrally woven with the woven graft tube at a first end and a second end such that warp from the woven graft tube is de-interlaced from the woven graft tube at the first end and re-interlaced with the woven graft tube at the second end;
supplemental warp is interlaced into the woven graft tube in replacement of the deinterlaced warp so as to maintain weave density in the woven graft tube under the plurality of woven extensions, and
the plurality of woven extensions and portions of the woven graft tube disposed directly under the plurality of woven extension have independent weft from one another; and
at least one stent wire disposed about the suture-free stent graft,
wherein the at least one stent wire is at least partially disposed between the woven graft tube and the plurality of woven extensions at a plurality of securement sites, securing the woven graft tube to the at least one stent wire.

10. The suture-free stent graft construct of claim 9, wherein the plurality of woven extensions include loop extensions integral with the woven graft tube at only the first end and the second end, defining a plurality of through-channels, and the at least one stent wire passes through the plurality of through-channels.

11. The suture-free stent graft construct of claim 9, wherein the plurality of woven extensions include slot extensions integral with the woven graft tube at the first end and the second end as well as along one edge extending from the first end to the second end, defining a plurality of pockets, and the at least one stent wire is partially disposed within the plurality of pockets.
